SEQUENCE LISTING (3 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 31 aa, Homo sapiens
EAEDLQVGQVELGGGPGAGSLQPLALEGSLQ

(19) United States
(12) Patent Application Publication
(10) Pub. No.: US 2026/0357194 A1
(43) Pub. Date: Nov. 12, 2026

(54) Optimization of Chemically Defined Cell Culture Medium to a Non-Diabetic State by Supplementation of C-Peptide Alongside Insulin

(71) Applicant: utR Biotech Limited, Winnipeg (CA)

(72) Inventor: David Petch, Winnipeg (CA)

(21) Appl. No.: 19/354,997

(22) Filed: Oct. 10, 2025

Related U.S. Application Data
(60) Provisional application No. 63/804,239, filed on May 12, 2025.

(51) Int. Cl. C12N 5/00; A61K 38/00; C07K 16/00; C12N 5/071

(52) U.S. Cl. CPC C12N 5/0018; A61K 38/00; C07K 16/00; C12N 5/0682; C12N 2500/10; C12N 2500/32; C12N 2500/34; C12N 2500/38; C12N 2501/33; C12N 2501/998

(57) ABSTRACT

A cell culture medium, kits, and methods for restoring physiological relevance by eliminating the artificial “diabetic” state typical of conventional media is described. The medium comprises a basal medium, insulin, a carrier protein (such as recombinant human serum albumin, cyclodextrin, or a synthetic polymer), and C-peptide. The concentration of C-peptide, ranging from 0.1 mg/L to 1.5 mg/L, is maintained in an approximately equimolar ratio with insulin, adjusted based on the medium's glucose concentration. This supplementation modulates coordinated signaling through the Insulin Receptor (IR), G-protein coupled receptor 146 (GPR146), and the Elastin Receptor Complex, leading to reduced cellular stress and improved metabolism. The invention enhances the production of therapeutic molecules (e.g., antibodies with reduced acidic charge variants) and improves the function of therapeutic cells (e.g., T-cells with a statistically significant reduction in exhaustion markers PD-1 and TIM-3). The carrier, present at a minimum concentration of 10 mg/L, ensures the bioavailability and stability of C-peptide in the medium.

### CROSS REFERENCES TO PRIORITY APPLICATION

The instant application claims the benefit of U.S. Provisional Patent Application 63/804,239, filed May 12, 2025, entitled “Optimization of Chemically Defined Cell Culture Media to a Non-Diabetic State by Supplementing with C-Peptide, Modulating Insulin Receptor, GPR146 and Relaxin Receptor Signaling, and Incorporating Advanced Culture Techniques”, the contents of which are incorporated herein by reference.

### SEQUENCE LISTING XML

Sequence Listing XML file entitled “uTR sequence listing.xml”, created Oct. 9, 2025, 3798 bytes, is incorporated herein by reference for all purposes.

### FIELD OF THE INVENTION

This invention relates to the field of cell culture, specifically to novel cell culture media, kits, and methods for improving the physiological relevance and productivity of cell cultures. The invention achieves this through the supplementation of media with C-peptide to modulate signaling through key pathways including the Insulin Receptor (IR), G-protein coupled receptor 146 (GPR146), and the Elastin Receptor Complex.

### BACKGROUND OF THE INVENTION

Cell culture is a foundational technology in modern biology and medicine. The evolution to chemically defined media (CDM) has improved consistency but introduced a potential flaw: current CDM formulations can create an artificial, “diabetic” state in vitro. This is because they typically include high, non-physiological levels of insulin without its co-secreted partner, C-peptide. Historically dismissed as an inert byproduct of insulin synthesis, C-peptide is now understood to be a pleiotropic hormone with essential biological functions (Liu et al., 2015; Pinger et al., 2017). Its universal omission from cell culture media creates a state of hormonal imbalance that can lead to aberrant cell metabolism. The inclusion of a carrier protein like recombinant human serum albumin (rHSA) is critical in CDM to ensure the transport and bioavailability of C-peptide, as C-peptide's activity often requires complex formation with albumin and zinc to be effective (Jacobs et al., 2022; Liu et al., 2015; Spence et al., 2020). The present invention represents a non-obvious inventive leap that addresses this long-standing gap in the art, which was missed for decades because conventional experimental approaches systematically underperform compared to modern AI-driven methods in identifying complex protein-ligand interactions (Abramson et al., 2024).

### SUMMARY OF THE INVENTION

The present invention provides a solution to the artificial diabetic state of conventional cell culture by restoring a key endocrine signal. The invention provides a cell culture medium comprising a basal medium supplemented with C-peptide, insulin, and a carrier protein, wherein the concentrations of C-peptide and insulin are maintained at a physiologically relevant ratio and are adjusted based on the glucose concentration of the medium. This optimized medium promotes coordinated signaling through key pathways, including the Insulin Receptor (IR), GPR146, and the Elastin Receptor Complex.

According to an aspect of the invention, there is provided a chemically defined cell culture medium for enhancing the production of a therapeutic molecule, the medium comprising: a. a basal cell culture medium; b. Insulin; c. C-peptide, wherein the C-peptide is present in an approximately equimolar ratio to the insulin; and d. a carrier selected from the group consisting of recombinant human serum albumin (rHSA), a cyclodextrin, a synthetic polymer, and combinations thereof.

According to another aspect of the invention, there is provided a cell culture medium for enhancing the production of a therapeutic molecule, the medium comprising: a. a basal cell culture medium; b. Insulin; c. C-peptide, wherein the C-peptide is present in an approximately equimolar ratio to the insulin; and d. a carrier selected from the group consisting of recombinant human serum albumin (rHSA), a cyclodextrin, a synthetic polymer, and combinations thereof.

According to another aspect of the invention, there is provided a method of producing a therapeutic molecule, the method comprising the step of: a. culturing a cell line that expresses the therapeutic molecule in the cell culture medium described above.

According to another aspect of the invention, there is provided a method of producing a therapeutic molecule, the method comprising the step of: a. culturing a cell lime comprising the therapeutic agent in the cell culture medium described above; and isolating the therapeutic agent from the cell culture medium.

According to another aspect of the invention, there is provided a method for adjusting C-peptide and insulin concentrations in a cell culture medium, comprising calculating the amounts to add using the formulae: Insulin to add (μg/L)=2.08-2.54 or 2.31×Glucose concentration (g/L) and C-peptide to add (μg/L)=1.33-161 or 1.47×Glucose concentration (g/L).

According to another aspect of the invention, there is provided a method for reducing fibrosis or inhibiting collagen type 4 synthesis in a cultured cell population, the method comprising culturing the cell population in the cell culture medium described above.

According to another aspect of the invention, there is provided a method for manufacturing a recombinant therapeutic product, comprising the steps of: (a) culturing a cell line in the medium described above; (b) isolating the therapeutic molecule from the medium; and (c) formulating the isolated therapeutic molecule into a drug product.

According to another aspect of the invention, there is provided a cell culture kit for preparing a physiologically relevant cell culture medium, the kit comprising: a. a bottle of basal cell culture medium; and b. a separate vial containing a concentrated supplement, the supplement comprising insulin, C-peptide, and a carrier protein, wherein the components are included with instructions to combine them to achieve a final C-peptide: insulin approximately equimolar ratio.

According to another aspect of the invention, there is provided a cell culture kit for preparing a cell culture medium, the kit comprising: a. a bottle of basal cell culture medium; and b. a first vial comprising insulin; a second vial comprising C-peptide, and a carrier; and instructions for combining the insulin and the C-peptide and the carrier to achieve a final C-peptide: insulin approximately equimolar ratio.

According to another aspect of the invention, there is provided a recombinant therapeutic molecule produced by the method described above, characterized by having a reduced level of acidic charge variants.

According to another aspect of the invention, there is provided a cellular or viral composition produced by a method comprising culturing cells in the medium described above, the composition being selected from the group consisting of: a therapeutic viral vector with enhanced infectivity; a population of differentiated stem cells with enhanced functional maturity; and a population of therapeutic CAR-T cells with a reduced exhaustion marker profile.

According to another aspect of the invention, there is provided a therapeutic cell population produced by the method described above.

### DETAILED DESCRIPTION OF PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a cell culture medium, kits, and methods for restoring physiological relevance by eliminating the artificial “diabetic” state typical of conventional media. The medium comprises a basal medium, insulin, a carrier protein (such as recombinant human serum albumin, cyclodextrin, or a synthetic polymer), and C-peptide. The concentration of C-peptide, ranging from 0.1 mg/L to 1.5 mg/L, is maintained in an approximately equimolar ratio with insulin, adjusted based on the medium's glucose concentration. This supplementation modulates coordinated signaling through the Insulin Receptor (IR), G-protein coupled receptor 146 (GPR146), and the Elastin Receptor Complex, leading to reduced cellular stress and improved metabolism. The invention enhances the production of therapeutic molecules (e.g., antibodies with reduced acidic charge variants) and improves the function of therapeutic cells (e.g., T-cells with a statistically significant reduction in exhaustion markers PD-1 and TIM-3). The carrier, present at a minimum concentration of 10 mg/L, ensures the bioavailability and stability of C-peptide in the medium.

According to an aspect of the invention, there is provided a chemically defined cell culture medium for enhancing the production of a therapeutic molecule, the medium comprising: a. a basal cell culture medium; b. Insulin; c. C-peptide, wherein the C-peptide is present in an approximately equimolar ratio to the insulin; and d. a carrier selected from the group consisting of recombinant human serum albumin (rHSA), a cyclodextrin, a synthetic polymer, and combinations thereof.

According to another aspect of the invention, there is provided a cell culture medium for enhancing the production of a therapeutic molecule, the medium comprising: a. a basal cell culture medium; b. Insulin; c. C-peptide, wherein the C-peptide is present in an approximately equimolar ratio to the insulin; and d. a carrier selected from the group consisting of recombinant human serum albumin (rHSA), a cyclodextrin, a synthetic polymer, and combinations thereof.

In some embodiments, the approximately equimolar ratio is a C-peptide: insulin molar ratio between 0.8:1 and 1.2:1.

As used herein, “insulin” refers to, collectively and individually as appropriate, mammalian insulin, for example, human insulin, porcine insulin or a synthetic equivalent insulin, that is, an artificial, synthetic insulin that functions approximately equivalently to for example human insulin or native insulin for the source of the cells being grown on the cell culture medium.

As used herein, “C-peptide” refers to, collectively and individually as appropriate, mammalian C-peptide, for example, human C-peptide, porcine C-peptide or a synthetic equivalent C-peptide, that is, an artificial, synthetic C-peptide that functions approximately equivalently to for example human C-peptide or native C-peptide for the source of the cells being grown on the cell culture medium.

In some embodiments of the invention, the human C-peptide peptide or protein comprises or consists of or consists essentially of the amino acid sequence as set forth in SEQ ID No: 3: EAEDLQVGQVELGGGPGAGSLQPLALEGSLQ. In some embodiments of the invention, the human insulin protein comprises or consists of or consists essentially of two chains, A and B, linked by disulfide bonds, having the amino acid sequences as set forth in SEQ ID No: 1 and SEQ ID No: 2, respectively: A-chain: GIVEQCCTSICSLYQLENYCN (SEQ ID No: 1) and B-chain: FVNQHLCGSHLVEALYLVCGERGFFYTPKT (SEQ ID No: 2).

In some embodiments, the concentration of C-peptide in the cell culture medium is between 0.1 mg/L and 1.5 mg/L.

In some embodiments, the concentration of insulin in the cell culture medium is between 0.19 mg/L and 2.88 mg/L

In some embodiments, the concentration in the cell culture medium of C-peptide is 0.52 mg/L and the concentration of insulin in the cell culture medium is 1 mg/L.

In some embodiments of the invention, the approximately equimolar ratio corresponds to a C-peptide to Insulin weight ratio between 0.509:1 and 0.763:1.

The carrier may be present at a concentration between 10 mg/L and 1000 mg/L.

The medium may further comprise a divalent metal cation, for example, zinc sulfate, heptahydrate.

In some embodiments, the concentration of the divalent metal cation is sufficient to facilitate Insulin hexamer formation with C-peptide-carrier complex formation in a molar ratio of 1:3 of zinc ions to insulin molecules.

As will be apparent to those of skill in the art, as used herein, ‘standard basal medium’ or ‘basal cell culture medium’ refers to a formulation comprising the essential nutrients necessary to support mammalian cell growth in vitro. A person of ordinary skill in the art of cell culture would understand a basal medium to include, at minimum: inorganic salts providing physiological ionic balance; a carbon and energy source; essential amino acids; a pH buffering system; and vitamins supporting cellular metabolism.

The basal medium may be supplemented with additional components such as trace elements, nucleosides, lipids, and other nutrients depending on the specific cell type and application. Commercial examples of suitable basal media will be readily apparent to those of skill in the art.

In preferred embodiments, the basal medium is serum-free and chemically defined, meaning all components are of known chemical composition and concentration, with no undefined biological materials such as fetal bovine serum or animal-derived hydrolysates.

In some embodiments, the basal cell culture medium comprises: inorganic salts providing physiological ionic balance; a carbon and energy source; essential amino acids; a pH buffering system; and vitamins supporting cellular metabolism.

In some embodiments, the concentrations of C-peptide and insulin are adjusted, for example, dynamically adjusted, based on the glucose concentration of the medium.

In some embodiments, the combination of C-peptide and insulin is present in an amount effective to modulate coordinated signaling through at least two receptors selected from the group consisting of the Insulin Receptor (IR), G-protein coupled receptor 146 (GPR146), and the Elastin Receptor Complex. For example, the signaling modulation may suppress the TGF-β/SMAD3 pathway, providing an anti-fibrotic effect.

According to another aspect of the invention, there is provided a method of producing a therapeutic molecule, the method comprising the step of: a. culturing a cell line that expresses the therapeutic molecule in the cell culture medium described above.

According to another aspect of the invention, there is provided a method of producing a therapeutic molecule, the method comprising the step of: a. culturing a cell lime comprising the therapeutic agent in the cell culture medium described above; and isolating the therapeutic agent from the cell culture medium.

According to another aspect of the invention, there is provided a method for adjusting C-peptide and insulin concentrations in a cell culture medium, comprising calculating the amounts to add using the formulae: Insulin to add (μg/L)=2.08-2.54 or 2.31×Glucose concentration (g/L) and C-peptide to add (μg/L)=1.33-161 or 1.47×Glucose concentration (g/L).

The cell line may be a Chinese Hamster Ovary (CHO) cell line.

The therapeutic molecule may be a monoclonal antibody or a recombinant protein.

The culturing step may result in an increase in final antibody titer of at least 10% compared to an identical medium lacking C-peptide.

In some embodiments, the resulting therapeutic molecule is characterized by a reduced level of acidic charge variants or less than 5% high molecular weight aggregates.

The insulin and C-peptide may be delivered to the culture as a single feed stream throughout the culture duration.

According to another aspect of the invention, there is provided a method for reducing fibrosis or inhibiting collagen type 4 synthesis in a cultured cell population, the method comprising culturing the cell population in the cell culture medium described above.

The cell line may be a therapeutic cell line selected from the group consisting of human or murine T-cells, Natural Killer (NK) cells, and stem cells.

In some embodiments, the culturing step results in a statistically significant reduction in the co-expression of exhaustion markers PD-1 and TIM-3.

In some embodiments, the culturing is performed in an advanced system selected from a 3D bioreactor, an organ-on-a-chip system, or on a biocompatible scaffold.

In some embodiments, cells are grown in the cell culture medium for modeling a disease state in vitro by adjusting the concentrations of glucose, C-peptide, and insulin to mimic a metabolic profile.

In some embodiments, the culturing step is used for modeling Alzheimer's disease in vitro by regulating the expression or activity of ADGRG1 on microglia.

In some embodiments, the method further comprises formulating the isolated therapeutic agent into a drug product.

According to another aspect of the invention, there is provided a method for manufacturing a recombinant therapeutic product, comprising the steps of: (a) culturing a cell line in the medium described above; (b) isolating the therapeutic molecule from the medium; and (c) formulating the isolated therapeutic molecule into a drug product.

According to another aspect of the invention, there is provided a cell culture kit for preparing a physiologically relevant cell culture medium, the kit comprising: a. a bottle of basal cell culture medium; and b. a separate vial containing a concentrated supplement, the supplement comprising insulin, C-peptide, and a carrier protein, wherein the components are included with instructions to combine them to achieve a final C-peptide: insulin approximately equimolar ratio.

According to another aspect of the invention, there is provided a cell culture kit for preparing a cell culture medium, the kit comprising: a. a bottle of basal cell culture medium; and b. a first vial comprising insulin; a second vial comprising C-peptide, and a carrier; and instructions for combining the insulin and the C-peptide and the carrier to achieve a final C-peptide: insulin approximately equimolar ratio.

The concentrated supplement (insulin and C-peptide: carrier) may be formulated as a lyophilized powder or as a stabilized liquid.

In some embodiments, the instructions direct the user to monitor glucose concentration and adjust the supplement addition based on a calculation protocol.

According to another aspect of the invention, there is provided a recombinant therapeutic molecule produced by the method described above, characterized by having a reduced level of acidic charge variants.

According to another aspect of the invention, there is provided a cellular or viral composition produced by a method comprising culturing cells in the medium described above, the composition being selected from the group consisting of: a therapeutic viral vector with enhanced infectivity; a population of differentiated stem cells with enhanced functional maturity; and a population of therapeutic CAR-T cells with a reduced exhaustion marker profile.

According to another aspect of the invention, there is provided a therapeutic cell population produced by the method described above.
1. Optimized Cell Culture Media Composition The invention is a novel cell culture medium formulation designed to mimic a non-diabetic physiological state.

Basal Medium: The composition begins with a standard basal medium.

C-Peptide: The basal medium is supplemented with human C-peptide to achieve a final concentration between 0.1 nM and 10 nM, with a preferred range of to 0.5 nm to 2.0 nM.

Insulin: The medium is further supplemented with human insulin, maintained at an equimolar concentration to C-peptide.

Carrier Requirement: The medium must be supplemented with a carrier, for example, a carrier protein, preferably recombinant human serum albumin (rHSA), at a minimum concentration of to facilitate transport and bioavailability, typically in a range of 10 mg/L to 1000 mg/L.

Alternative Carriers: In alternative embodiments, the carrier may be selected from stabilizing molecules such as cyclodextrins or biocompatible synthetic polymers. These alternatives function by providing a stabilizing non-covalent binding environment for C-peptide that prevents degradation and aggregation, thereby ensuring its bioavailability and biological activity in a manner functionally analogous to that provided by rHSA.
2. Glucose-Dependent Adjustment Protocol To maintain physiological relevance, the concentrations of C-peptide and insulin are dynamically adjusted based on the glucose concentration of the basal medium, which is typically between 2.0 g/L and 8.0 g/L.

The amount of C-peptide to add should be the molar equivalent of the amount of insulin added. The required amounts of insulin and C-peptide are calculated as follows:

Insulin to add (μg/L)=2.08-2.54×Glucose concentration (g/L), for example, 2.31×Glucose concentration (g/L).

C-peptide to add (μg/L)=1.33-1.61×Glucose concentration (g/L), for example, 1.47×Glucose concentration (g/L).

Molar Equivalence Calculation Example: To achieve an equimolar ratio, one must account for the difference in molecular weights (Insulin: 5808 g/mol; C-peptide: ~3696 g/mol). For example, if 1 mg of insulin is added to 1 liter of medium:

Moles of Insulin=(0.001 g)/(5808 g/mol)=1.72×10⁻⁷ mol.

Equimolar Moles of C-Peptide=1.72×10⁻⁷ mol.
Mass of C-Peptide=(1.72×10⁻⁷ mol)×(3696 g/mol)=0.000636 g=0.636 mg.

Therefore, for every 1 mg of insulin, ~0.636 mg of C-peptide should be added to achieve an equimolar ratio.
3. Enhanced Mechanism of Action: Modulation of Key Receptor Signaling The addition of C-peptide beneficially modulates three key receptor pathways:

Insulin Receptor (IR) Modulation: C-peptide binds to an allosteric site on the IR, potentiating insulin's effect. This leads to more efficient glucose uptake and anabolic signaling, allowing for the use of lower, more physiological insulin concentrations

GPR146 Modulation: C-peptide is a novel GPR146 agonist (Yosten et al., 2013).

Elastin Receptor Modulation: C-peptide binding stimulates the angiotensin II type 2 receptor (AT2R), activating the protective arm of the renin-angiotensin system (RAS) and suppressing the TGF-β/SMAD3 pathway, which provides anti-fibrotic effects (Li et al., 2018).

### 4. Applications of the Invention

The present invention has broad applications in various fields, including:

Disease Modeling: Facilitating the creation of more accurate in vitro models of human diseases, particularly for studying cutting-edge neuro-immune pathways such as the role of ADGRG1 on microglia in Alzheimer's disease.

Drug Discovery: Enabling the screening of drug candidates that target C-peptide signaling pathways and evaluating new therapies.

Biopharmaceutical Production: Enhancing the production of therapeutic proteins, such as monoclonal antibodies, by optimizing cell growth and productivity.

Cell-Based Therapies: Improving the efficacy and safety of therapies like stem cell and CAR-T cell therapies by promoting cell survival, differentiation, and function.

Regenerative Medicine: Enhancing tissue and organ regeneration by promoting cell proliferation and differentiation, supported by C-peptide's anti-fibrotic effects via suppression of the TGF-β/SMAD3 pathway.

Toxicology Testing: Evaluating the toxicity of compounds in a more physiologically relevant environment for more accurate safety assessments.

### 5. Manufacturing and Quality Control

The production of C-peptide supplemented media requires meticulous attention to component stability, compatibility, and sterility.

Base Medium Preparation: The process begins with the precise formulation of the base medium, with careful adjustment of pH (7.2-7.4) and osmolality (280-320 mOsm/kg), followed by sterile filtration.

Component Solution Preparation: A solution of rHSA (at least 10 mg/L) is prepared, to which purified recombinant C-peptide is added. A separate solution of insulin is prepared in acidified sterile water (pH 2-3) to maintain solubility.

Final Integration: The C-peptide-albumin complex is added to the base medium first with gentle stirring, followed by the sequential addition of the insulin solution. Final pH and osmolality are re-verified.

Quality Control Testing: Testing is performed, including sterility testing, endotoxin evaluation (<0.1 EU/mL), functional verification via growth promotion testing, and stability testing, ensuring less than 5% degradation of C-peptide activity over 30 days at 2-8 °C.

The invention will now be further explained and/or elucidated by way of examples; however, the invention is not necessarily limited to or by the examples.

### EXAMPLES

Example 1: Enhanced Production of Monoclonal Antibodies in CHO Cells Chinese Hamster Ovary (CHO) cells are cultured in a 3 L fed-batch bioreactor under four conditions: Control (commercial CDM), Inventive Medium (CDM+rHSA+glucose-dependent insulin/C-peptide), C-Peptide Only, and Inventive Medium Minus Carrier. The inventive medium is expected to show a ~40% increase in final antibody titer and a significant reduction in cellular stress compared to the control. The “Minus Carrier” condition is expected to show no significant improvement, demonstrating the carrier protein is an essential component.

Example 2: Justification of the C-Peptide Concentration Range Mesenchymal stem cells (MSCs) are cultured with varying concentrations of C-peptide (0 to 20.0 nM). Cell viability is measured, and it is expected that viability will show a statistically significant increase at 0.1 nM, peak between 1.0 and 5.0 nM, and remain high at 10.0 nM, supporting the claimed effective range.

Example 3: Efficacy of a Synthetic C-Peptide Analogue Primary human hepatocytes are cultured under three conditions: Control, rHSA System (inventive medium), and Synthetic System (using a synthetic C-peptide analogue). It is expected that both the “rHSA System” and the “Synthetic System” will show a significant increase in albumin production, demonstrating that synthetic analogues fall within the scope of the invention.

Example 4: Enhanced Expansion and Reduced Exhaustion of CAR-T Cells Human CAR-T cells are cultured in a standard T-cell medium (Control) versus the Inventive Medium. It is expected that cells in the “Inventive Medium” will exhibit significantly higher viable cell expansion and a statistically significant reduction in the expression of exhaustion markers like PD-1 and TIM-3, demonstrating the utility in producing more potent therapeutic cells.

While the preferred embodiments of the invention have been described above, it will be recognized and understood that various modifications may be made therein, and the appended claims are intended to cover all such modifications which may fall within the spirit and scope of the invention.

### REFERENCES

1. Abramson J, et al. Accurate structure prediction of biomolecular interactions with AlphaFold 3. Nature. 2024; 630 (8016): 493-500.
2. Jacobs M J, Geiger M K, Summers S E, DeLuca C P, Zinn K R, Spence D M. Albumin Glycation Affects the Delivery of C-Peptide to the Red Blood Cells. ACS Meas Sci Au. 2022; 2 (3): 278-286.
3. Li Y, Zhong Y, Gong W, Gao X, Qi H, Liu K, et al. C-peptide prevents SMAD3 binding to alpha promoters to inhibit collagen type 4 synthesis. J Mol Endocrinol. 2018; 61 (1): 47-56.
4. Liu Y, Chen C, Summers S, Medawala W R, Spence D M. C-peptide and zinc delivery to erythrocytes requires the presence of albumin: implications in diabetes explored with a 3D-printed fluidic device. Integr Biol. 2015; 7 (5): 534-543.
5. Piao X, et al. G-protein-coupled receptor ADGRG1 drives a protective microglial state in Alzheimer's disease through MYC activation. Neuron. 2025; [epub ahead of print].
6. Pinger C W, Entwistle K, Bell T M, Liu Y, Spence D M. C-peptide replacement therapy in type 1 diabetes: are we in the trough of disillusionment? Mol Biosyst. 2017; 13 (8): 1432-1437.
7. Spence D M, Patel H, Nolan B, Keyzers R, Libs P, White B, et al. A C-peptide complex with albumin and Zn2+increases measurable GLUT1 levels in membranes of human red blood cells. Sci Rep. 2020; 10 (1): 17493.
8. Turoczi T, Sasi V, Ghadge G, Zsengeller Z, Hinds C, et al. C-peptide corrects blood-brain barrier dysfunction in a mouse model of type 1 diabetes. Diabetologia. 2019; 62 (3): 512-524.
9. Yosten G L C, Kolar G R, Rediger J N, Samson W K. The insulin C-peptide is a novel GPR146 agonist. Am J Physiol Regul Integr Comp Physiol. 2013; 305 (6): R642-R648.

1. A cell culture medium for enhancing the production of a therapeutic molecule, the medium comprising: a. a basal cell culture medium; b. Insulin; c. C-peptide, wherein the C-peptide is present in an approximately equimolar ratio to the insulin; and d. a carrier selected from the group consisting of recombinant human serum albumin (rHSA), a cyclodextrin, a synthetic polymer, and combinations thereof.

2. The medium of claim 1, wherein the approximately equimolar ratio is a C-peptide: insulin molar ratio between 0.8:1 and 1.2:1.

3. The medium of claim 1, wherein the concentration of C-peptide is between 0.1 mg/L and 1.5 mg/L.

4. The medium of claim 1, wherein the concentration of insulin is between 0.19 mg/L and 2.88 mg/L.

5. The medium of claim 1, wherein the concentration of C-peptide is 0.52 mg/L and the concentration of insulin is 1 mg/L.

6. The medium of claim 1, wherein the approximately equimolar ratio corresponds to a C-peptide to Insulin weight ratio between 0.509:1 and 0.763:1.

7. The medium of claim 1, wherein the carrier is present at a concentration between 10 mg/L and 1000 mg/L.

8. The medium of claim 1, further comprising a divalent metal cation.

9. The medium of claim 8 wherein the divalent metal cation is zinc sulfate, heptahydrate.

10. The medium of claim 9 wherein at a concentration of the divalent metal cation is sufficient to facilitate Insulin hexamer formation with C-peptide-carrier complex formation in a molar ratio of 1:3 of zinc ions to insulin molecules.

11. The method of claim 1 wherein the basal cell culture medium comprises: Inorganic salts providing physiological ionic balance; a carbon and energy source; essential amino acids; a pH buffering system; and vitamins supporting cellular metabolism.

12. The medium of claim 1 wherein zinc sulfate heptahydrate is added to the medium at about 16.4 mg/L per 1 mg/mL of insulin.

13. The medium of claim 1, wherein the concentrations of C-peptide and insulin are adjusted based on the glucose concentration of the medium.

14. A method of producing a therapeutic molecule, the method comprising the step of: a. culturing a cell lime comprising the therapeutic agent in the cell culture medium of claim 1; and isolating the therapeutic agent from the cell culture medium.

15. The method of claim 14, wherein the cell line is a Chinese Hamster Ovary (CHO) cell line.

16. The method of claim 14, wherein the therapeutic molecule is a monoclonal antibody or a recombinant protein.

17. The method of claim 14, wherein the insulin and C-peptide are delivered to the culture as a single feed stream throughout the culture duration.

18. The method according to claim 14, further comprising formulating the isolated therapeutic agent into a drug product.

19. A cell culture kit for preparing a cell culture medium, the kit comprising: a. a bottle of basal cell culture medium; and b. a first vial comprising insulin; a second vial comprising C-peptide, and a carrier; and instructions for combining the insulin and the C-peptide and the carrier to achieve a final C-peptide: insulin approximately equimolar ratio.

20. The kit of claim 19, wherein the insulin and the C-peptide and carrier are formulated as a lyophilized powder or as a stabilized liquid.
